(19) United States
(12) Patent Application Publication
(10) Pub. No.: US 2018/0236011 A1
(43) Pub. Date: Aug. 23, 2018

(54) METHODS TO ENHANCE TUMOR-TARGETING BY BACTERIA

(71) Applicant: AntiCancer, Inc., San Diego, CA (US)

(72) Inventors: Kei Kawaguchi, San Diego, CA (US); Robert M. Hoffman, La Jolla, CA (US)

(21) Appl. No.: 15/902,769

(22) Filed: Feb. 22, 2018

Related U.S. Application Data
(60) Provisional application No. 62/462,165, filed on Feb. 22, 2017.

(51) Int. Cl. A61K 35/74; A61K 31/495; A61K 31/437; A61P 35/00

(52) U.S. Cl. CPC A61K 35/74; A61P 35/00; A61K 31/437; A61K 31/495

(57) ABSTRACT

Methods using chemotherapy drugs promote targeting of S. typhimurium A1-R of melanoma, thereby enhancing efficacy against the melanoma PDOX. A metastatic melanoma obtained from the right chest wall of a patient was previously established orthotopically in the right chest wall of nude mice as a patient-derived orthotopic xenograft (PDOX) model. We previously showed that the combination of tumor targeting Salmonella typhimurium A1-R (S. typhimurium A1-R) and chemotherapy was highly effective against the melanoma PDOX. In the present study, we investigated the mechanism of the high efficacy of this combination. Combination therapy significantly increases S. typhimurium A1-R tumor targeting alone (S. typhimurium A1-R+TEM: p<0.01, S. typhimurium A1-R+VEM: p<0.01).

### CROSS REFERENCE TO RELATED APPLICATION

This application claims priority to United States (Provisional) Patent Application to Kawaguchi, entitled “METHODS TO ENHANCE TUMOR-TARGETING OF BACTERIA,” application No. 62/462,165, filed Feb. 22, 2017, now pending, the disclosure of which is hereby incorporated entirely herein by reference.

### BACKGROUND OF THE INVENTION

### Technical Field

This invention relates to methods for the enhancing tumor targeting of bacteria. In particular, the invention relates to methods of using chemotherapy drugs to enhance tumor-targeting by S. typhimurium bacteria.

### State of the Art

D The tumor-targeting Salmonella typhimurium A1-R (S. typhimurium A1-R), developed by our laboratory, is auxotrophic for Leu-Arg, which prevents it from mounting a continuous infection in normal tissues. S. typhimurium A1-R was effective against primary and metastatic tumors as monotherapy in nude mouse models of major cancers, including prostate, breast, lung, pancreatic, ovarian stomach, and cervical cancer. In addition, S. typhimurium A1-R was effective against patient-derived orthotopic models (PDOX) of pancreatic cancer, sarcoma and melanoma.

### DISCLOSURE OF EMBODIMENTS OF THE INVENTION

Embodiments of the present invention include methods for enhancing bacterial targeting of tumors. The foregoing and other features and advantages of the invention will be apparent to those of ordinary skill in the art from the following more particular description of the invention and the accompanying drawings.

Disclosed is a method for treating cancer comprising steps administering a chemotherapy medication to mammal with a tumor; inoculating the mammal with a bacterium; and enhancing infection of a cell of the tumor with the bacterium in response to the chemotherapy medication.

In some embodiments, the chemotherapy mediation is temozolamide. In some embodiments, the chemotherapy medication is vemurafenib.

In some embodiments, the bacterium is of the genus Salmonella. In some embodiments, the bacterium is Salmonella typhimurium.

In some embodiments, the bacterium is an A1-R auxotroph of Salmonella typhimurium. In some embodiments, the tumor is a malignant melanoma. In some embodiments, the tumor is a sarcoma. In some embodiments, the tumor is an adenocarcinoma. In some embodiments, the tumor is a squamous cell carcinoma.

### BRIEF DESCRIPTION OF THE DRAWINGS

FIG. 1 is a flowchart showing steps of a method for enhancing tumor-targeting by bacteria.

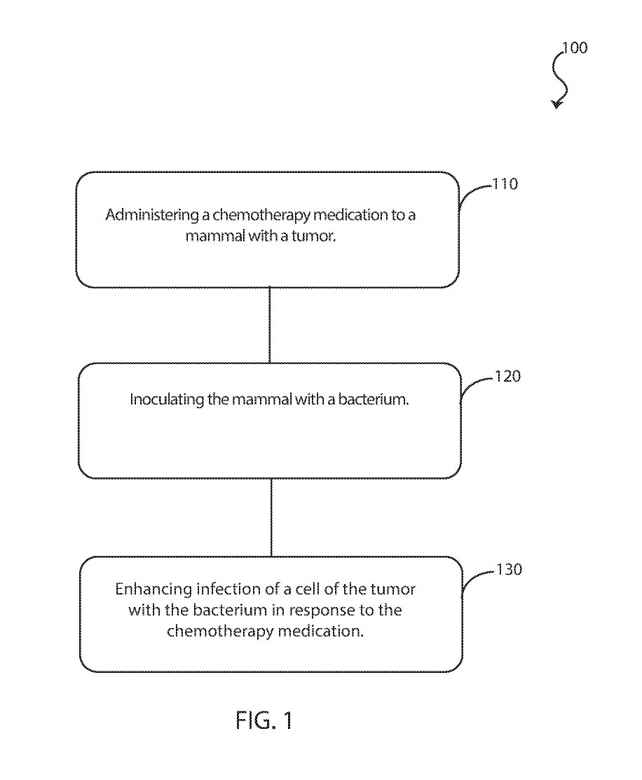

### DETAILED DESCRIPTION OF EMBODIMENTS OF THE INVENTION

As mentioned herein above, the disclosed invention relates to methods for enhancing bacterial targeting of tumors. In particular, the invention relates to methods of using chemotherapy drugs to enhance tumor-targeting by S. typhimurium bacteria.

In the present study, we demonstrate that chemotherapy greatly enhances S. typhimurium A1-R tumor targeting. All treatments significantly inhibited tumor growth compared to untreated control (S. typhimurium A1-R: p<0.01; TEM combined with S. typhimurium A1-R: p<0.01; VEM combined with S. typhimurium A1-R: p<0.01; on day 14 after initiation. Combination therapy with S. typhimurium A1-R was significantly more effective than S. typhimurium A1-R alone with TEM: p<0.01; with VEM: p<0.01. Confocal microscopy showed that the S. typhimurium A1-R could directly target the melanoma PDOX. Targeting ability for melanoma of S. typhimurium A1-R was evaluated by S. typhimurium A1-R-GFP fluorescent area. Both VEM and TEM significantly increase S. typhimurium A1-R-GFP fluorescent area compared to S. typhimurium A1-R alone (TEM: p<0.01; VEM: p<0.01).

The histology of the original patient tumor and the untreated PDOX tumor were similar, containing the same types of cells. VEM and TEM caused extensive necrosis in the tumor when each was combined with S. typhimurium A1-R and much more extensive than S. typhimurium A1-R. Salmonella typhimurium (VNP20009) has been previously used for effective therapy of a melanoma. VNP20009 was attenuated by a lipid A-mutation (msbB), purine auxotrophy (purl) and amino acid auxotrophy. The tumor-targeting S. typhimurium A1-R, developed by our laboratory, is auxotrophic only for Leu-Arg and is less attenuated.

We show that both VEM and TEM promoted S. typhimurium A1-R tumor targeting with elevated accumulation of S. typhimurium A1-R that led to extensive tumor necrosis. This is the first report to elucidate the mechanism by which combination of chemotherapy with S. typhimurium A1-R is extremely effective.

Despite progress in melanoma therapy, there is still no cure for stage III and IV disease due to drug resistance, tumor heterogeneity and an immunosuppressive tumor environment. In addition, the presence of melanin appears to interfere with chemotherapy and radiotherapy of this recalcitrant disease. The present results suggest that S. typhimurium A1-R could potentiate chemotherapy for melanoma. Clinical trials are warranted for this strategy.

Previously-developed concepts and strategies of highly-selective tumor targeting can take advantage of molecular targeting tumors, including tissue-selective therapy with focuses on unique differences between normal and tumor tissues. The combination of chemotherapy with S. typhimurium A1-R was highly effective on a chemotherapy-resistant melanoma in a PDOX mouse model. This treatment strategy has important future clinical potential, which possibly can be realized in the near future.

### Mice

Athymic nu/nu nude mice (AntiCancer Inc., San Diego, Calif.), 4-6 weeks old, were used in this study. Animals were housed in a barrier facility on a high efficacy particulate arrestance (HEPA)-filtered rack under standard conditions of 12-hour light/dark cycles. The animals were fed an autoclaved laboratory rodent diet. All mouse surgical procedures and imaging were performed with the animals anesthetized by subcutaneous injection of a ketamine mixture (0.02 ml solution of 20 mg/kg ketamine, 15.2 mg/kg xylazine, and 0.48 mg/kg acepromazine maleate). The response of animals during surgery was monitored to ensure adequate depth of anesthesia. The animals were observed on a daily basis and humanely sacrificed by CO₂ inhalation if they met the following humane endpoint criteria: severe tumor burden (more than 20 mm in diameter), prostration, significant body weight loss, difficulty breathing, rotational motion and body temperature drop. All animal studies were conducted in accordance with the principles and procedures outlined in the National Institutes of Health Guide for the Care and Use of Animals under Assurance Number A3873-1.

### Patient-Derived Tumor

A 75-year-old female patient was previously diagnosed with a melanoma of the right chest wall. The tumor was resected in the Department of Surgery, University of California, Los Angeles (UCLA). Written informed consent was provided by the patient, and the Institutional Review Board (IRB) of UCLA approved this experiment. Establishment of PDOX models of melanoma by surgical orthotopic implantation (SOI) After nude mice were anesthetized with the ketamine solution described above, a 5-mm skin incision was made on the right chest into the chest wall, which was split to make space for the melanoma tissue fragment. A single tumor fragment was implanted orthotopically into the space to establish the PDOX model. The wound was closed with a 6-0 nylon suture (Ethilon, Ethicon, Inc., NJ, USA).

Preparation and Administration of S. typhimurium A1-R

GFP-expressing S. typhimurium A1-R bacteria (AntiCancer Inc.,) were grown overnight on LB medium (Fisher Sci., Hanover Park, Ill., USA) and then diluted 1:10 in LB medium. Bacteria were harvested at late-log phase, washed with PBS, and then diluted in PBS. S. typhimurium A1-R was injected intravenously. A total of 5×10₇ CFU S. typhimurium A1-R in 100 μl PBS was administered to each mouse.

Treatment Study Design in the PDOX Model of Melanoma

PDOX mouse models were randomized into four groups of 10 mice each: untreated control (n=10); treated with S. typhimurium A1-R (5×10₇ CFU/100 μl, i.v., q_{w}×2, n=10); treated with TEM (25 mg/kg, p.o., qd×14) combined with S. typhimurium A1-R (5×10₇ CFU/100 i.v., qw×2, n=10); treated with VEM (30 mg/kg, p.o., qd×14) combined with S. typhimurium A1-R (5×10⁷CFU/100 μl, i.v., qw×2, n=10). Tumor length and width were measured twice a week. Tumor volume was calculated with the following formula: Tumor volume (mm3)=length (mm)×width (mm)×width (mm)×½. Data are presented as mean±SD. The tumor volume ratio is defined at the tumor volume at any given time point relative to the initial tumor volume.

### Confocal Microscopy

The FV1000 confocal microscope (Olympus, Tokyo, Japan) was used for high-resolution imaging. Fluorescence images were obtained using the 20×/0.50 UPLAN FLN and 40×/1.3 oil Olympus UPLAN FLN objectives. The tumor fluorescent area was analyzed with UVP software (UVP, Upland, Calif.).

### Histological Analysis

Fresh tumor samples were fixed in 10% formalin and embedded in paraffin before sectioning and staining. Tissue sections (5 μm) were deparaffinized in xylene and rehydrated in an ethanol series. Hematoxylin and eosin (H&E) staining was performed according to standard protocols. Histological examination was performed with a BHS System Microscope (Olympus Corporation, Tokyo, Japan). Images were acquired with INFINITY ANALYZE software (Lumenera Corporation, Ottawa, Canada).

### Statistical Analysis

JMP version 11.0 was used for all statistical analyses. Significant differences for continuous yariables were determined using the Mann-Whitney U test. Line graphs expressed average values and error bar showed SD. A probability value of P≤0.05 was considered statistically significant.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above.

What is claimed is:

1. A method for treating cancer comprising steps:
administering a chemotherapy medication to mammal with a tumor;
inoculating the mammal with a bacterium; and
enhancing infection of a cell of the tumor with the bacterium in response to the chemotherapy medication.

2. The method of claim 1, wherein the chemotherapy mediation is temozolamide.

3. The method of claim 1, wherein the chemotherapy medication is vemurafenib.

4. The method of claim 1, wherein the bacterium is of the genus Salmonella.

5. The method of claim 1, wherein the bacterium is Salmonella typhimurium.

6. The method of claim 1, wherein the bacterium is an A1-R auxotroph of Salmonella typhimurium.

7. The method of claim 1, wherein the tumor is a malignant melanoma.

8. The method of claim 1, wherein the tumor is a sarcoma/

9. The method of claim 1, wherein the tumor is an adenocarcinoma.

10. The method of claim 1, wherein the tumor is a squamous cell carcinoma.
